(19) United States
(12) Patent Application Publication
(10) Pub. No.: US 2009/0166222 A1
(43) Pub. Date: Jul. 2, 2009

(54) ELECTRICAL NANOTRAPS FOR SPECTROSCOPICALLY CHARACTERIZING BIOMOLECULES WITHIN

(75) Inventors: Chad A. Mirkin, Wilmette, IL (US); Gengfeng Zheng, Evanston, IL (US); Lidong Qin, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: NORTHWESTERN UNIVERSITY, Evanston, IL (US)

(21) Appl. No.: 12/206,057

(22) Filed: Sep. 8, 2008

Related U.S. Application Data
(60) Provisional application No. 60/993,010, filed on Sep. 7, 2007.

(51) Int. Cl. C12Q 1/68; G01N 27/26

(52) U.S. Cl. 205/777.5; 204/403.01

(57) ABSTRACT

A method that combines on-wire-lithography (OWL) nanogaps, an electric field concentrating technique, and surface enhanced Raman spectroscopy (SERS) is disclosed for sensitive detection of analytes with small sample sizes in a chip format.

### CROSS-REFERENCE TO RELATED APPLICATIONS

This application claims the benefit of U.S. Provisional Application No. 60/993,010, filed Sep. 7, 2007, which is incorporated by reference in its entirety herein.

### STATEMENT OF GOVERNMENT INTEREST

This invention was made with U.S. government support under Air Force Material Command Law Office/JAZI Grant No. FA8650-06-C-7617 and National Science Foundation/NSEC Grant No. EEC-0647560. The government has certain rights in this invention.

### BACKGROUND

Sensitive detection of chemical and biological species with low dose sample sizes is highly desired in recently developed micro-array technology, micro-fluidic devices, and other micro-sensing systems (MacBeath, Nature Genetics 32:526-532 (2002); Stone, et al., Annual Review of Fluid Mechanics 36:381-411 (2004); Roco, et al., Current Opinion in Biotechnology 14:337-346 (2003); Ferrari, Nature Reviews Cancer 5:161-171 (2005)). Preferred for the development of new sensors is high efficiency signal transduction associated with selective recognition of a species of interest, and fast mass-transport process of analytes towards the miniaturized sensor devices (Nair, et al., Applied Physics Letters 88, (2006)). In addition, the detection system is expected to be simple and easy to integrate, and the signal should be not only strong, but accurate, specifically with fingerprint information (Cao, et al., Science 297:1536-1540 (2002)). Significant research progress has been made in sensor systems based on fluorescence (Wilson, et al. Angewandte Chemie-International Edition 45:6104-6117 (2006)), Raman spectroscopy (Yan, et al. Sensors and Actuators B-Chemical 121:61-66 (2007)), quantum dots (Gao, et al., Nature Biotechnology 22:969-976 (2004)), nanoparticles (Rosi, et al., Chemical Reviews 105:1547-1562 (2005)), and electrical (Zheng, et al., Nature Biotechnology 23:1294-1301 (2005) and Bakker, et al., Analytical Chemistry 78:3965-3983 (2006)) and mechanical devices (Shekhawat, et al. Science 311:1592-1595 (2006)). However, a biosensing system having all the features listed above has not yet been realized. Thus, a need exists for a biosensing system having such features.

### SUMMARY

Disclosed herein are methods of detecting the presence or concentration of an analyte using nanowires capable of having an electric field applied across them. More specifically, disclosed herein is a method of assaying for the presence of concentration of an analyte or plurality of analytes in a sample comprising contacting the sample with a nanowire, applying an electrical field across the nanowire, and detecting the analyte by measuring a detection event signal having a signal intensity, wherein the signal intensity is correlated to the presence or concentration of the analyte in the sample. In some cases, the signal intensity of the detection event is greater than the signal intensity in the absence of applying an electrical field.

The nanowire comprises at least one nanodisk array comprising at least two nanodisks separated by a gap, each nanodisk independently having a thickness of about 20 nm to about 1 μn, and the gap being about 2 nm to about 1 μm. The nanowire is connected to two electrodes such that an electric field can be applied to the nanowire. In some embodiments, each nanodisk has a thickness of about 20 nm to about 500 nm. In various embodiments, the gap is about 2 nm to about 500 nm.

In some embodiments, the analyte is a charged analyte. In various embodiments, the analyte is a nucleic acid, a protein, a peptide, a carbohydrate, a lipid, a cell, a bacteria, a virus, or a mixture thereof. In some embodiments, the analyte further comprises a fluorescent label or a Raman label.

In some embodiments, the nanowire is modified to further comprise a detection reagent. The detection reagent can be within the gap of the nanowire, on the surface of a nanodisk, or both. In various cases, the detection reagent comprises a label, such as a fluorescent label or a Raman label. The detection reagent can be a target for the analyte. In one specific example, the analyte is a nucleic acid, and the detection reagent is a complementary nucleic acid. In other cases, the analyte is a protein or antibody, and the detection reagent is a ligand for the protein or an antigen of the antibody.

In another aspect, disclosed herein are apparatuses having a nanowire connected to an electrode, capable of detecting analytes.

### BRIEF DESCRIPTION OF THE DRAWINGS

FIG. 1A shows a nanowire with the analyte localized in the gap of the nanowire, and the analyte has a Raman label.

FIG. 1B shows a schematic of surface functionalization of gaps and hybridization with DNA analytes, where an alkylthiol terminated oligonucleotide can be directly linked to the gold (Au) nanodisks on either side of the gap, and/or coupled to the silica coating within the gap, when the silica is pre-modified with 3-aminopropyl trimethoxysilane. The target oligonucleotides modified with Raman (left) or fluorescence dyes (right) are trapped inside the gaps when an AC electric field is applied.

FIG. 2A shows a scanning electron microscopy (SEM) image of synthesized nanorods. The central Ni portion is etched away to form gap structures. FIGS. 2B and 2C show SEM and corresponding fluorescence images, respectively, of a nanowire described herein showing the localization of a Cy5-labeled DNA in the gap. The gap position is highlighted by the white arrow. FIG. 2D shows the line profile of the fluorescence intensity, as indicated by the white dot line in FIG. 2C, both with and without an applied AC field, where the fluorescent signal is only measured when the AC filed is applied. Scale bars in 2A, 2B, and 2C are 500 nm, 1 μm, and 1 μm, respectively.

FIG. 3A shows an SEM image of the as-synthesized nanorods. The central Ni portion is etched away to form gap structures. FIG. 3B shows an SEM image of a nanowire contacting with electrodes, where the gap is noted with a white arrow and the gap size is 50 nm. FIG. 3C shows a Raman spectrum of the Cy5-labeled DNA target. FIGS. 3D and 3E show scanning Raman microscopy images indicating that Raman intensities are significantly increased due to the electric field concentrating of target DNA into the nanogaps that function as Raman hot spots. The scale bars in 3A, 3B, 3D and 3E are 200 nm, 2 μm, 1 μm and 1 μm, respectively.

FIG. 4A shows scanning Raman microscopy images of nanowires under an applied electric field in the presence of various concentrations of DNA analyte. The scale bar is 1 μm. FIG. 4B shows a comparison of Raman intensity as a function of target concentration with (right bar) and without (left bar) an applied electric field.

### DETAILED DESCRIPTION

Recent development of the surface enhanced Raman scattering (SERS) effect (Nie, et al. Science 275:1102-1106 (1997) and Haynes, et al., Analytical Chemistry 77:338 A-346A (2005)) based on optimized nanostructures, such as nanoparticles, nanowires, and the on-wire-lithography (OWL) nanogaps/nanodisks, enables the enhancement of Raman signal up to 8-14 orders of magnitude, which provides a promising means to achieve high sensitivity and accuracy in chemical and biological detection. See Kneipp, et al., Physical Review Letters 78:1667-1670 (1997); Wang, et al., Journal of the American Chemical Society 127:14992-14993 (2005); Tao, et al., Nano Letters 3:1229-1233 (2003); Qin, et al., Science 309:113-115 (2005); Qin, et al., Proceedings of the National Academy of Sciences of the United States of America 103:13300-13303 (2006); and Qin, et al., Nano Lett. 7:3849-3853 (2007)).

Despite many advances achieved by SERS-based and other detection techniques, the sensor's capability is still far from optimal. Two intrinsic factors that limit the sensing performance are the mass-transport rate of analytes from bulk solution towards sensor surface (Myszka, et al., Biophysical Chemistry 64:127-137 (1997)), as well as the binding equilibrium between analytes and immobilized ligands on the sensor surface (Karlsson, et al., Journal of Immunological Methods 145:229-240 (1991)).

Various approaches such as electrical field concentrating techniques have been developed to expedite the mass-transport rate as well as increase the effective analyte concentration near the active sensor element (Edman, et al., Nucleic Acids Research 25:4907-4914 (1997); Gurtner, et al., Electrophoresis 23:1543-1550 (2002); Chou, et al., Biophysical Journal 83:2170-2179 (2002); Hoettges, et al., Journal of Physics D-Applied Physics 36:L101-L104 (2003); Green, et al., Physical Review E 61:4011-4018 (2000); Holzel, et al., Physical Review Letters 95 (2005); Wong, et al., Analytical Chemistry 76:6908-6914 (2004)). For example, negative oligonucleotides can be attracted towards the electrode surface biased with positive voltages based on an electrophoretic effect. However, as the whole electrode area is under a uniform potential, this method does not favor concentrating and sensing of low dose samples.

Recently, the AC electrokinetic manipulation of biomolecules has been of interest. The non-uniform AC electric fields generated by microelectrode produce steady fluid flow in electrolytic solutions, and result in an enhanced concentration of biomolecules inside the electrode gaps where the highest strength electric field exists.

Disclosed herein is a new material for biosensing which combines optimized SERS nanostructures, device integration, and AC electric field concentrating methods to achieve analyte detection with μL sample size and 230 fM concentration. The Raman signal of labeled analytes is detected on the nanowires, as these nanogaps can enhance the Raman coupling effect with fingerprint identity. In addition, by applying an AC electric field over the two ends of the nanowires, the analytes can be concentrated inside the nanogaps such that both the mass-transport rate and the effective analyte concentration near the sensor are significantly raised. Importantly, the positions where analytes are concentrated are overlapped with the positions of the highest SERS efficiency, thus sensitivity is significantly improved.

### Nanowires

On-wire lithograph (OWL) is used to prepare the nanowires used in the disclosed detection methods. OWL methods are described in International Patent Publication WO 2007/064390, and U.S. Ser. No. 11/372,583, now U.S. Pat. No. ______. As used herein, “nanorods” refers to small structures that are less than 10 μm, and preferably less than 5 μm, in any one dimension and that have a length to width ratio greater than one. The nanorods used in the present invention are multicomponent in nature. As used herein, “multicomponent” refers to an entity that comprises more than one type of material. For example, a multicomponent nanorod refers to a nanorod having sections of different materials, e.g., a nanorod with one or more Au segments and one or more nickel (Ni) segments.

The metal component of the nanorod can be any metal compatible with in situ electrochemical deposition. Examples of such metals include, but are not limited to, indium-tin-oxide, titanium, platinum, titanium tungstide, gold, silver, nickel, copper, and mixtures thereof.

A “nanowire,” interchangeably referred to as a “gapped nanowire,” is a nanorod that has been subjected to etching to remove certain metal segments and leave behind others. These nanowires have electronic properties that can be tailored from their compositional components (i.e., the identities of the metals forming the nanorod). The use of metals having different chemical and electrical properties allows the creation of gaps in these nanowires when the nanowire is treated with a solution that dissolves one metal of the nanorod while the other metal is unaffected.

A nanodisk array is a series of metal segments (i.e., nanodisks) separated by a gap. In some cases, the gap is between about 2 nm and about 500 nm. Other gap ranges contemplated include in the range of about 5 and about 160 nm, about 10 to about 120 nm, about 15 to about 100 nm, about 20 to about 75 nm, or about 25 to about 50 nm. Specific examples of gap sizes include 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 nm. In other cases, the gap is greater than 500 nm. Gaps up to an including 2 μm may also be incorporated into a nanodisk array.

The metal segments remaining after etching form nanodisks. Disk thicknesses for nanodisks include, but are not limited to, ranges of about 20 nm to about 500 nm, about 40 nm to about 250 nm, and about 50 nm to about 120 nm. Specific disk thickness contemplated for use in the present invention include 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, and 500 nm. In some cases, the disk thickness of the nanodisk is at least 500 nm and can be up to 5 μm.

A series of nanodisk arrays having different characteristics (e.g., disk thickness and gap size) may be present on the same nanowire. Separation of the nanodisk arrays on a nanowire is achieved using separation gaps. The length of a separation gap is dependent upon the size of the nanodisk array. Typically, a separation gap is at least two times greater, preferably three times greater, than the total length of a nanodisk array. For example, a nanodisk array composed of two 120 nm disks separated by a 50 nm gap can be separated from a second nanodisk array by a separation gap of about 1 μm. For nanodisk arrays having larger disk thickness and gaps, larger separation gaps are needed.

The number of gaps in a nanodisk array can vary. At least one gap is present in a nanodisk array. Gaps numbering from 1, 2, 3, 4, 5, 6, 7, 8, 9, and 10, or more, can all be incorporated into a nanodisk array. The number of gaps in a nanodisk array determines the number of nanodisks in the array. For example, one gap correlates to two nanodisks; two gaps correlate to three nanodisk; and three gaps to four nanodisks.

As used herein, the term “sacrificial metal” refers to a metal that can be dissolved under the proper chemical conditions. Examples of sacrificial metals include, but are not limited to, nickel which is dissolved by a strong acid such as nitric or hydrochloric acid, and silver which is dissolved by nitric acid or a methanol/ammonia/hydrogen peroxide mixture.

As used herein, the term “etching” refers to a process of dissolving a sacrificial metal segment using conditions suitable for dissolving or removing the metal comprising the sacrificial segment. As mentioned above, such etching solutions include, but are not limited to, hydrochloric or nitric acid and a methanol/ammonia/hydrogen peroxide mixture.

As used herein, “coating” refers to a material that is positioned to contact one side of a multicomponent nanorod, prior to the etching step. The purpose of the coating is to provide a bridging substrate to hold segments of the etched nanorod (i.e., a nanowire) together after removal of the intervening sacrificial metal segments in the etching process. Thus, a gap of a nanowire contains exposed coating. Nonlimiting examples of coatings used in this invention include a gold/titanium alloy and silica. A coating with a gold/titanium alloy allows for the nanowire to conduct an electrical current, whereas a silica coating can electrically isolate the various nanodisk arrays from each other. Other backings may be chosen to provide other electrical, chemical, or physical characteristics to the nanowire, depending upon the end use of the nanowire.

The surfaces of nanodisks are clean, i.e., free from contamination of stabilizing surfactants or other organic chemicals, because the OWL synthetic process uses nitric acid which removes essentially all organic compounds from the surface of the nanodisks. This clean surface allows for better functionalization and also decreases Raman scattering noise attributed to surface contaminants. Detection of small analyte concentrations or probe molecules therefore is enhanced due to the decreased scattering noise and tailorable functionalization of the nanodisks.

Different metals can be incorporated into the nanodisks by simple modifications to the synthesis. Nonlimiting examples of metals that can be incorporated include silver (Ag), gold (Au), and copper (Cu), which are particularly useful as SERS substrates. SERS substrates are interchangeably referred to as SERS active substrates herein. A suitable SERS substrate is the disclosed nanowires.

Detection of an analyte is via a detection event. The detection event is typically fluorescence or Raman spectroscopy, but can be by any means that produces a measurable change. Detection can proceed either directly or in combination with a detection reagent. In certain cases, the analyte does not have an appreciable fluorescence or Raman scattering cross section, and a detection reagent, e.g., a label, is needed to provide sufficient fluorescence or Raman scattering for detection.

The label can be a moiety having one or more of the following properties: (a) a strong absorption band in the vicinity of an excitation wavelength (extinction coefficient near 10⁴ or greater); (b) a functional group which will enable it to be covalently or non-covalently bound to an analyte of interest; (c) photostability; (d) sufficient surface and resonance enhancement to allow detection limits of at least 10 μg, and preferably in the subnanogram range; (e) minimal exhibition of strong fluorescence emission at the excitation wave length used, usually denoted as having a large Stokes shift; and (f) a relatively simple scattering pattern with a few intense peaks. When more than one label is used, it is preferred that the labels having spectral patterns which do not interfere with one another, e.g., overlap, so several indicator molecules can be analyzed simultaneously. In some embodiments, spectral overlap is a desired characteristic because the emission spectrum from one label can overlap the excitation spectrum of another, exciting the first label and resulting in a “pumping” of the second.

Examples of fluorescent and Raman labels include, but are not limited to, cyanine dyes (e.g., Cy3 and Cy5), 4-(4-aminophenylazo)phenylarsonic acid monosodium salt, arsenazo I, basic fuchsin, Chicago sky blue, direct red 81, disperse orange 3,2-(4-hydroxyphenylazo)benzoic acid (HABA), erythrosin B, trypan blue, ponceau S, ponceau SS, 1,5-difluoro-2,4-dinitrobenzene, methylene blue (MB), and p-dimethylaminoazobenzene (PMA).

In some embodiments, the detection reagent can be covalently attached to the analyte of interest. In other embodiments, the detection reagent can be non-covalently attached to the analyte of interest, e.g., via hybridization, pi-stacking, hydrogen bonding, van der Waals interactions, chelation, and the like. In still others, the nanowires themselves can be functionalized with detection reagents that can be attached to the surface of the nanodisks and/or to a surface on the coating, which is exposed in the gaps of the nanowire. In such cases, the detection reagent typically comprises a functionality that allows for its immobilization on the surface of the nanodisk or on the coating of the gap.

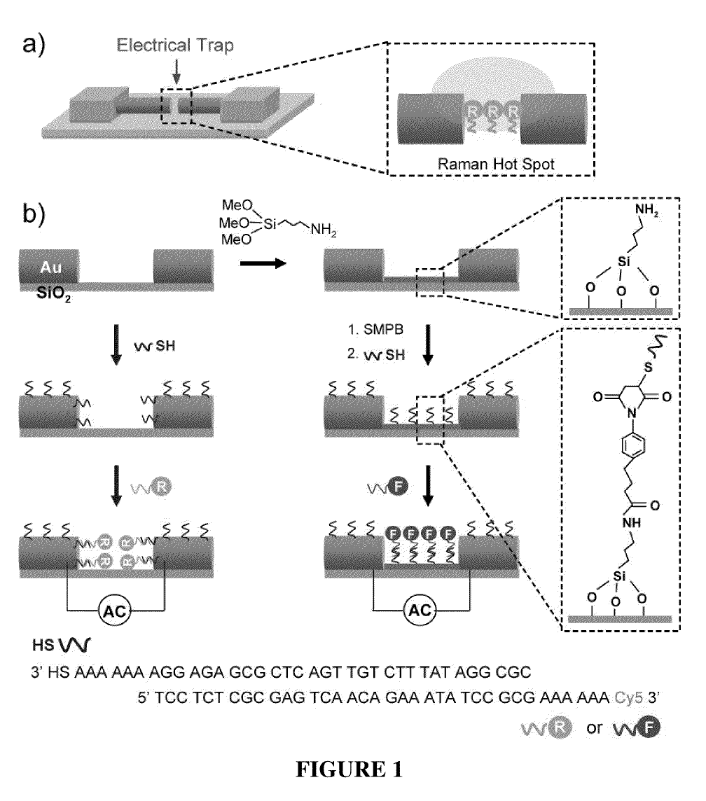

For example, a detection reagent comprising a thiol moiety can be appended to the surface of a gold nanodisk, such as a thiol-nucleic acid. For cases where the detection reagent is attached to the coating of the gap, the coating is typically silica, and the silica is modified with a functionalized silane, e.g. an amino silane such as the aminoalkylsilane 3-aminopropyltrimethoxysilane. The amino moiety can then be used to append a detection reagent to the coating of the gap, e.g., any detection reagent having a functional group capable of reacting with an amino moiety, such as a carboxylic acid, a succinimide, an anhydride, or the like. Two specific examples of appending detection reagent to the nanowires are use of thiolated DNA probes via amino-silane and succinimidyl 4-(p-maleimidophenyl) butyrate (SMPB), or directly functionalized with thiolated DNA probes (see, e.g., FIG. 1B).

In some embodiments, the detection reagent is capable of interacting with the analyte. Some non-limiting examples of such interactions include non-covalent interactions (e.g., van der Waals interactions, hydrogen-bonding, hybridization, and the like) or covalent interactions. For example, when the analyte comprises a nucleic acid, the detection reagent can comprise a complementary nucleic acid or a ligand of a nucleic acid. When the analyte comprises a protein, the detection reagent can comprise a ligand of the protein.

In some embodiments, more than one detection reagent is used. For example, a detection reagent having a fluorescent label or Raman label can be used for enhanced detection, and the surface of a nanodisk or surface between a gap can be modified with a detection reagent.

The AC electric field is applied to the two ends of the nanowire through the metal electrode leads for concentrating the target analytes inside the nanowire gaps. The electric field can concentrate the analyte to the gap of the nanowire, which can lead to an increase in intensity of a detection event signal. For example, for a charged analyte, the analyte will be attracted to the electric field and concentrated in the gap. The resulting fluorescence or Raman signal will be more pronounced than in the absence of the electric field. This phenomenon can allow for increased sensitivity of detection of low concentration analytes, down to about 230 fM. Other concentrations of analyte that can be detected using the disclosed methods include at least about 1 pM, at least about 10 pM, and at least about 1 nM.

All patents, publications and references cited herein are hereby fully incorporated by reference. In case of conflict between the present disclosure and incorporated patents, publications and references, the present disclosure should control.

### EXAMPLES

The following examples are provided to illustrate the invention, but are not intended to limit the scope thereof.

### Synthesis of Nanowires

An Anodisc® anodic aluminum oxide (AAO) membrane (Whatman International Ltd.) was used as template for electrodeposition of multi-segmented nanorods. First, 100 nm of silver was thermally evaporated onto one side of the AAO template, which served as a cathode in a three-electrode electrochemical cell. The cell also contained a Pt counter electrode and an Ag/AgCl reference electrode. Commercially available Orotemp 24RTU (for Au) and Nickel Sulfamate SEMI Bright RTU (for Ni) electroplating solutions (Technic. Inc.) were used for electrochemical deposition. Different segments of the nanorods (gold, nickel, and gold) were electrochemically deposited at a constant potential (Au: −900 mV, Ni: −850 mV, vs. Ag/AgCl), and the lengths of the nanorod sections were tailored by varying the amount of charge passing through the electrodes. See U.S. Pat. No. 7,422,696. After electrodeposition, the silver backing and AAO template were dissolved with a mixture of methanol, concentrated aqueous ammonia, and 30% hydrogen peroxide (volume ratio: 4:1:1), and 3 M sodium hydroxide solutions, respectively. The nanorods were collected and dispersed onto glass slides, and transferred into a plasma-enhanced chemical vapor deposition chamber (PECVD, PlasmalabμP, Plasma Technology Inc.). The slides were heated to 300° C., and SiH₄/N₂ (10%/90%) and N₂O gases were introduced into the chamber at flow rates of 40 sccm and 40 sccm, respectively. The throttle pressure was 200 mTorr, and the RF power was 12 Watts for 5 min. The corresponding silica deposition rate was 10 nm/min. Next, the nanorods were mixed with 50% hydrochloric acid for 4 hours to etch away the middle nickel section, then re-dispensed in ethanol and deposited onto a silicon substrate with 600-nm-thick thermal oxide. The electrodes connecting to the nanorods were defined by electron beam lithography (Quanta-600), followed by thermal evaporation of 10 nm of Cr and 400 nm of Au.

### Functionalization of Nanowires

The Si/SiO₂ wafer with nanogap devices was first treated with piranha solution for 15 min, and then incubated with 3-aminopropyltrimethoxy silane (1% in water) solution for 2 hours. The silicon wafer was then rinsed with deionized water, cured at 120° C. for 10 min, and incubated overnight (about 8 to about 12 hours) with 0.1 M succinimidyl 4-(p-maleimidophenyl) butyrate (SMPB) solution in DMSO in the dark at room temperature. Afterwards, the wafer was rinsed with DMSO/ethanol then ethanol, and incubated with 100 μM DNA (SEQ ID NO: 1: 5′ CGC GGA TAT TTC TGT TGA CTC GCG AGA GGA AAA AAA SH 3′) in the coupling buffer (0.3 M NaCl, 10 mM phosphate buffer, 0.1% Tween-20, pH 7.4) for at least 12 hours.

### Electrical Concentrating Technique

A microfluidic channel was fabricated by sandwiching a 100-μm-thick plastic spacer between the silicon wafer substrate and a thin microscope cover slip. The target DNA (SEQ ID NO: 2: 5′ TCC TCT CGC GAG TCA ACA GAA ATA TCC GCG AAA AAA Cy5 3′) was dissolved in the hybridization buffer (20 mM NaCl, 10 mM phosphate buffer, 0.1% Tween-20, pH 7.4) and delivered through the microfluidic channel. An AC electric field was applied to the nanowire devices by a function generator (Agilent Inc., conditions were set at Vₚ₋ₚ=0.8-1.2 V, f=1-10 kHz). Typically, the DNA concentrating effect is observed within 10 min.

### Fluorescence, Raman and SEM Measurements

Fluorescence measurements were carried out using a fluorescence microscope (Model Axiovert 220M, Zeiss Inc.). The bright field images were first taken in order to locate the positions of the nanowire devices, and then the fluorescence images were subsequently recorded at the same position for image comparison. Raman spectra and images were recorded with a confocal Raman microscope (Model CRM200, WITec Inc.) equipped with a piezo-scanner and 100× microscope objectives (NA=0.90, Nikon Inc.). The spatial resolution is 400 nm. Each pixel in the images was constructed by integrating the Raman intensity of the main spectral peaks between 650 and 1450 cm⁻¹. The SEM images were recorded using a scanning electron microscope (Model LEO1525, Leo Inc.).

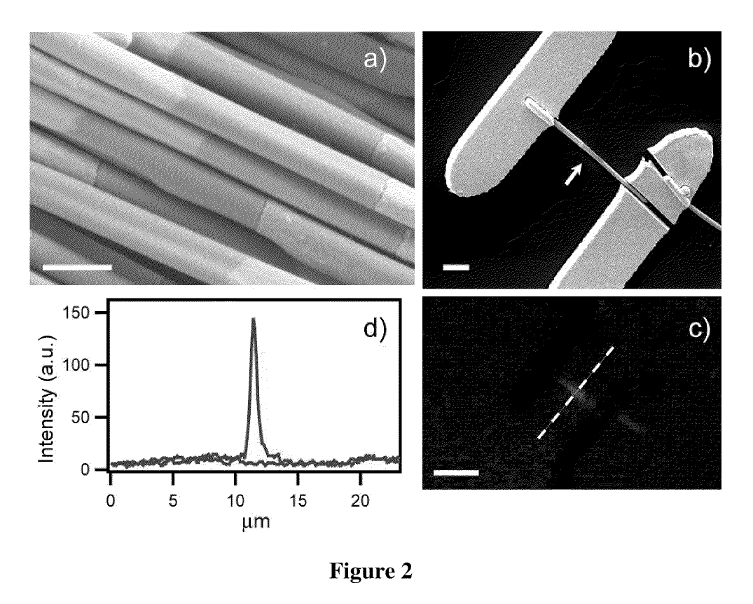

Fluorescence: The efficiency of OWL nanogap devices for electrical trapping and optical detection of biomolecules can be characterized using fluorescence. In a typical experiment, shown in FIG. 2, the OWL nanorods (with Au, gap, Au lengths 3, 1, 3 μm, respectively) were deposited onto silicon wafer surface and connected to metal electrodes by EBL, and then the nanorods were functionalized with the DNA probes (sequence: 5′ CGC GGA TAT TTC TGT TGA CTC GCG AGA GGA AAA AAA SH 3′; SEQ ID NO: 1). A solution containing 1 nM of the target DNA molecules (sequence: 5′ TCC TCT CGC GAG TCA ACA GAA ATA TCC GCG AAA AAA Cy5 3′; SEQ ID NO: 2) was delivered onto the nanowire surface via a microfluidic channel, with the channel volume around 1 μL. The salt concentration in the bulk buffer solution was tuned to around 10-20 mM. This low salt concentration is important for this electrical field enhanced DNA detection based on two reasons. Low-ionic strength buffers interfere less with the concentration of the charged analytes in the gaps of the nanowires (Bhatt, et al., Langmuir 21:6603-6612 (2005)). Low salt concentration also disfavors formation of spontaneous hybridization of DNA analytes in the sample solution. An AC electric field applied to the nanowires provided enhanced fluorescence intensities, which can be observed for nanogaps as small as 5 nm. There are several features of this experiment. First, the fluorescence intensity on the whole surface is low, suggesting the unfavorable hybridization conditions for DNA target molecules due to the low salt concentration. Hence even in the conditions with low concentration and volume of target molecules, the majority of the target DNA molecules can still be utilized and bound to the active sensor area. In this experiment the volume of solution used was about 1 μL, which equaled to the volume of the microfluidic channel and could be further reduced. Second, only the nanowires having an applied AC electric field resulted in fluorescence signal enhancement, while the nanowire with no AC electric field did not yield any measurable fluorescence enhancement over the background. FIGS. 2C and 2D show the line profiles across a nanowire and the resulting fluorescence intensity with and without an applied AC electric field. This data clearly demonstrates that the electric field has efficiently increased the local DNA concentrations into the nanogaps by trapping the DNA analyte inside the gaps. The strongest fluorescence intensity in the nanowire comes from the positions where a gap exists, instead of the whole area between the two metal electrode leads.

Arrays of microelectrodes with interfacing sharp protrudes were fabricated by photolithography and modified with thiolated DNA probes, and a similar AC electric field was applied to each pair of electrodes to concentrate the Cy5-DNA targets. The sequences of DNA probes and targets were SEQ ID NO: 1 and 2, respectively. It demonstrated that the highest fluorescence intensities were located inside the electrode gaps with shape protrude, as those shape interfacing electrode protrudes provide the highest electric field. In the other experiment, the target DNAs were conjugated with 40 nm-diameter Au nanoparticles. The SEM image showed that these DNA-nanoparticles were almost completely concentrated inside the protruding electrode gaps. These observations indicate that the analytes are concentrated into the positions where the highest electric field exists.

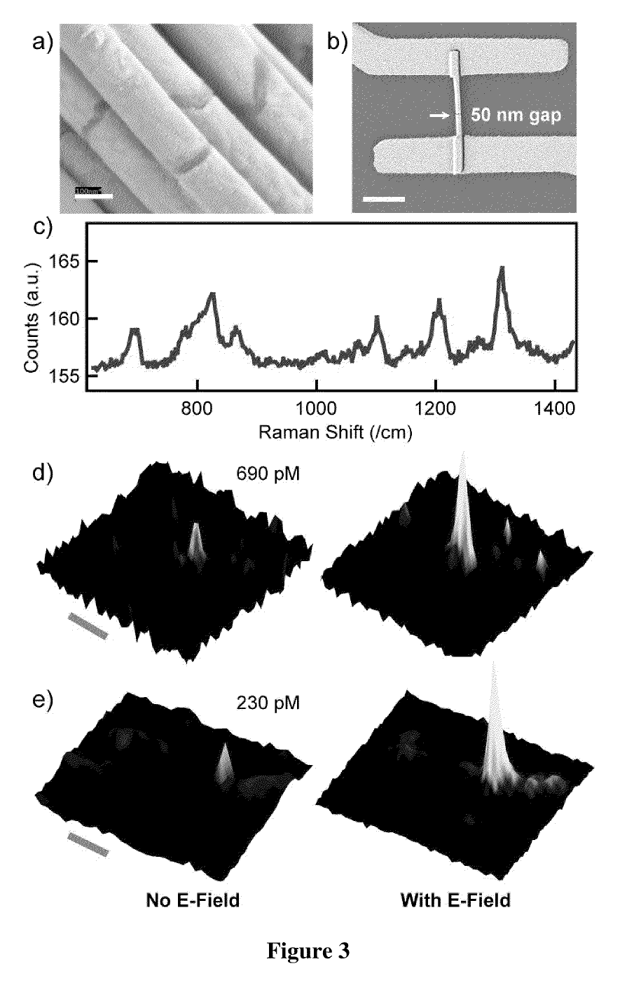

Raman: The Raman detection of oligonucleotides by nanowire sensors was carried out using similar device fabrication and surface functionalization approach as that for the fluorescence measurement, while the signal was readout by a confocal scanning Raman microscope. FIGS. 3A and 3B show SEM images of a nanowire (Au-gap-Au), in which the gap size was about 50 nm. This selection of gap size was based on prior studies on the gap size dependent Raman signal coupling efficiency, which shows the much higher Raman coupling effect with gap sizes under 100 nm. The AC electric field can be applied via the two metal leads contacting to each gold side of this nanorod, where the center gap portion (as highlighted by the white arrow in the figure) can be used to detect the Raman signal coupling of the target DNAs. A typical Raman spectrum of the Cy5-linked oligonucleotide sequence (SEQ ID NO: 2) on the nanowire surface is shown in FIG. 3C. The scanning Raman microcopy measurement was recorded by scanning a selected area of interest, where each pixel was constructed by integrating the entire spectral intensity from 700 to 1800 cm⁻¹, with the peak around 1500-1600 cm⁻¹ being excluded. (This highest peak signal around 1500-1600 cm⁻¹ is due to the superimposition of the Raman bands of silicon oxide wafer surface.)

To compare the Raman intensity enhancement with electric field concentrating to that when no electric field is applied, the same nanowires were approached twice under different measurement conditions. In the first run, the nanowire surface was incubated with target DNAs (SEQ ID NO: 2) in normal ionic strength buffer (0.3 M NaCl, 10 mM phosphate buffer, 0.1% Tween20, pH 7.4) for 24 hrs without electric field applied, and then was measured for the Raman signal. After the first Raman measurement, the chip was immersed into 80° C. deionized (DI) water for 10-15 min to fully dehybridize the bound target DNA molecules from the nanorod surface. Then, the sensor was re-hybridized with target DNAs by applying AC electric field and incubating in reduced ionic strength buffer (10 mM NaCl, 10 mM phosphate buffer, 0.1% Tween20, pH 7.4). The hybridization time can be reduced to about 1 hour due to the elevated mass-transport rate under the AC electric field. Then the Raman signals of the same OWL devices were re-measured. FIGS. 3D and 3E show the scanning Raman microscopy images (3-dimensional) from two different nanowires, in both conditions of without and with applying an electric field. The Raman signal inside the nanogap appears as a fully-resolved bright peak against a dark, smooth background as little Raman signal is recorded from the flat chip surface. Even when no electric field was applied, the central position of the nanogaps showed a peak of Raman signal, representing the surface enhanced Raman signal from the nanogap structure. When the electric field was applied, the local concentration of the target molecules was significantly increased. In the meantime these target analytes were driven to the nanogaps where the highest Raman coupling efficiency existed, therefore a much higher Raman peak was observed. From the two concentrations of target DNAs (690 pM and 230 pM), the Raman signal intensity increased about 40 and 20 times in total integration of the peak area as a result of the applied field, respectively. This experiment demonstrates that the integrated electric concentrating technique can substantially enhanced the Raman detection signal using the nanowires.

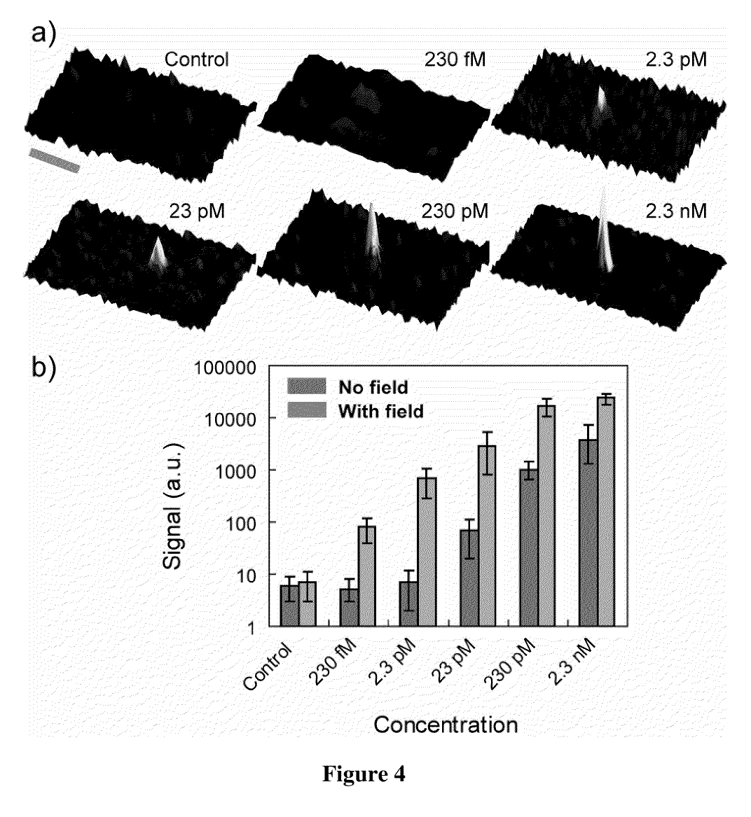

The sensitivity limit of DNA detection using these integrated electrical nanowires was measured under the same experimental conditions described for the Raman experiment above. FIG. 4A shows the measured scanning Raman microscopy images from different concentrations of target DNA molecules (SEQ ID NO: 2) by electric field driven nanowires, and the statistics was summarized in FIG. 4B, (comparing to that measured from similar devices but without AC electric field concentrating). The lowest measurable DNA concentration was around 230 fM, which is over 20 times better than previous Raman detection results (5 pM), and also at least two times better than the Tip-enhanced-Raman spectroscopy (TERS) technique (Dieringer, et al., Faraday Discuss. 132:9-26 (2006)). Furthermore, the method disclosed herein has several advantages over TERS: a simple setup, fast detection speed, and applicable in extending to a multiplexing level. This result can be further improved by optimizing the electric field parameters, designing different nanorod materials/structures, or using nanoparticle-functionalized DNAs for signal magnification.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

What is claimed:

1. A method of assaying for a presence or a concentration of an analyte or a plurality of analytes in a sample comprising:
a) providing a nanowire comprising at least one nanodisk array comprising at least two nanodisks, each nanodisk independently having a thickness of about 20 nm to about 5 μm, and at least one gap of about 2 nm to about 1 μm, said nanowire contacted to two electrodes;
b) contacting the nanowire with the sample;
c) applying an electrical current across the nanowire; and
d) detecting the analyte by measuring a detection event signal having a signal intensity, wherein the signal intensity is correlated to the presence or concentration of the analyte in the sample.

2. The method of claim 1, wherein the analyte is a charged analyte.

3. The method of claim 1, wherein the analyte is selected from the group consisting of a nucleic acid, a protein, a peptide, a carbohydrate, a bacteria, a virus, and a cell

4. The method of claim 1, wherein the analyte further comprises a fluorescent label or a Raman label.

5. The method of claim 1, wherein a detection reagent is present (1) within at least one gap of the nanowire, (2) on at least one nanodisk, or (3) both (1) and (2).

6. The method of claim 5, wherein the detection reagent comprises a fluorescent label or a Raman label.

7. The method of claim 5, wherein the detection reagent is capable of interacting with the analyte.

8. The method of claim 7, wherein the analyte comprises a nucleic acid and the detection reagent comprises a complementary nucleic acid.

9. The method of claim 5, wherein detection reagent comprises a fluorescent label and the signal is a fluorescence signal.

10. The method of claim 5, wherein the detection reagent comprises a Raman label and the signal is a surface enhanced Raman scattering signal.

11. The method of claim 1, wherein the signal intensity is greater than a signal intensity in the absence of applying an electrical current.

12. The method of claim 1, wherein the analyte concentration in the sample is less than 1 nM.

13. The method of claim 12, wherein the analyte concentration is less than 1 pM.

14. The method of claim 13, wherein the analyte concentration is less than 500 fM.

15. An apparatus comprising a nanowire having at least one nanodisk array comprising at least two nanodisks, each nanodisk independently having a thickness of about 20 nm to about 5 μm, and at least one gap of about 2 nm to about 1 μm, said nanowire in contact with two electrodes.

16. The apparatus of claim 15, wherein the at least one gap is about 25 to about 50 nm.

17. The apparatus of claim 15, further comprising a detection reagent on at least one nanodisk.

18. The apparatus of claim 17, wherein the detection reagent comprises a nucleic acid, a protein, a peptide, an antibody, a carbohydrate, a lipid, a cell, a bacteria, a virus, or a mixture thereof.

19. The composition of claim 15, wherein the at least one gap is about 25 to about 50 nm and each nanodisk has a thickness of about 100 to about 150 nm.


SEQUENCE LISTING (2 sequences)

SEQ ID NO: 1 - DNA, 36 bp, Artificial Sequence
  Synthetic poly nucleotide
cgcggatatttctgttgactcgcgagaggaaaaaaa

SEQ ID NO: 2 - DNA, 36 bp, Artificial Sequence
  Synthetic polynucleotide
tcctctcgcgagtcaacagaaatatccgcgaaaaaa